(19) United States
(12) Patent Application Publication
(10) Pub. No.: US 2011/0014223 A1
(43) Pub. Date: Jan. 20, 2011

(54) MULTIMERIC PROTEINS ABLE TO INDUCE AN ANTIBODY RESPONSE AGAINST THE BETA-AMYLOID AND USE THEREOF

(75) Inventors: Antonella Prisco, Roma (IT); Piergiuseppe De Berardinis, Roma (IT); Diana De Falco, Roma (IT); Antonella Caivano, Roma (IT)

Correspondence Address: LUCAS & MERCANTI, LLP, 475 PARK AVENUE SOUTH, 15TH FLOOR, NEW YORK, NY, 10016, US

(73) Assignee: CONSIGLIO NAZIONALE DELLE RICERCHE, Roma (IT)

(21) Appl. No.: 12/863,117

(22) PCT Filed: Jan. 27, 2009
(86) PCT No.: PCT/IB09/50329

§ 371 Date: Jul. 15, 2010

(30) Foreign Application Priority Data
Jan. 28, 2008 (IT) NA2008A000006

(51) Int. Cl. A61K 39/07; C12N 9/10; A61K 38/45; A61P 25/28

(52) U.S. Cl. 424/190.1; 435/193; 424/94.5

(57) ABSTRACT

The present invention relates to the fields of molecular biology, immunology and medicine. Peptide sequences that belong to the beta amyloid peptide are exposed on the surface of a multimeric structure derived from the E2 component of alpha keto acid dehydrogenase. The molecules are useful for the production of vaccines for the treatment and/or prevention of Alzheimer's disease.

### TECHNICAL FIELD OF THE INVENTION

The present invention relates to molecules with multimeric structure i.e. virus-like particles (VLP) based on the E2 component of the alpha-ketoacid dehydrogenase, compositions comprising them and uses thereof to stimulate an immune response against the beta amyloid peptide for the prevention and/or treatment of Alzheimer's disease.

### STATE OF THE ART

Alzheimer's disease, which affects over 12 million people throughout the world, is characterized by the deposition, in the brain, of insoluble protein aggregates, the amyloid plaques (Morgan et al., 2004). The main constituent of the plaques is the beta amyloid peptide (Aβ), which derives from the proteolytic processing of the APP (Amyloid precursor protein) membrane protein (Morgan et al., 2004). In animal models of Alzheimer's disease, it has been demonstrated that immunization with the beta amyloid peptide can reduce the quantity of cerebral amyloid and the progression of the pathology.

This effect is mediated by anti beta amyloid antibodies. (Morgan and Gitter, 2004; Weksler et al., 2005)

For the development of immunotherapy protocols of Alzheimer's disease, two important problems must be solved: the poor immunogenicity of the beta amyloid peptide, and the potential dangerousness of cell-mediated self-immune responses against the beta amyloid. In a clinical immunization trial of patients affected by Alzheimer's diseases with the entire beta amyloid peptide pre-aggregated, out of 300 individuals who received the vaccine only 59 exhibited an antibody response against beta amyloid with a titer above 1:2200, and 6% of patients developed an adverse inflammatory reaction, characterized by the infiltration of T lymphocytes in the brain (Gilman et al., 2005).

Anti-amyloid vaccines that consist of B epitopes of beta amyloid physically bound to a carrier that includes exogenous T epitopes are potentially more immunogenic and less dangerous for man than vaccines that contain T epitopes of beta amyloid (and hence autologous), that can trigger a cell-mediated self-immune reaction. The intensity of the humoral response to a recombinant vaccine based on the fusion of a carrier protein and of a specific epitope depends on the carrier, on the selected epitope, and also on the amino acidic residues that flank the epitope in the recombinant protein (Esposito et al., 2008), which makes it important to screen new constructs for immunogenicity optimization. The carrier used also influences the isotype of the antibody response (Chackerian et al., 2006), which is a correlate of the TH1/TH2 polarization of the T lymphocytes during the immune response. In the case of Alzheimer's disease, it has been hypothesized that vaccines that trigger a TH2 immune response are less dangerous than vaccines that trigger a TH1 (pro-inflammatory) response (Kim et al., 2007).

### DESCRIPTION OF THE INVENTION

The present invention consists of fusion proteins that contain an epitope B of beta amyloid (in particular, the epitope 1-11, DAEFRHDSGYE (SEQ ID NO. 6) or the epitope 2-6, AEFRH (SEQ ID NO.5)) and the catalytic domain of the E2 component of pyruvate dehydrogenase of Geobacillus stearothermophilus with the sequence:

(SEQ ID NO. 7)
PGPAAAEEKAAPAAAKPATTEGEFPETREKMSGIRRAIAKAMVHSKHTAP
HVTLMDEADVTKLVAHRKKFKAIAAEKGIKLTFLPYVVKALVSALREYPV
LNTSIDDETEEIIQKHYYNIGIAADTDRGLLVPVIKHADRKPIFALAQEI
NELAEKARDGKLTPGEMKGASCTITNIGSAGGQWFTPVINHPEVAILGIG
RIAEKPIVRDGEIVAAPMLALSLSFDHRMIDGATAQKALNHIKRLLSDPE
LLLMEA.

These recombinant proteins, expressed in Escherichia coli and purified, self-assemble in vitro to form the virus-like particles Aβ(1-11)E2₆₀ₘₑᵣ (Aβ(1-11)E2₆₀ₘₑᵣ) e Aβ(2-6)E2₆₀ₘₑᵣ (Aβ(2-6)E2₆₀ₘₑᵣ) which are used to trigger an antibody response against beta amyloid in laboratory mice. The antibody response obtained after two injection of the virus-like particles reaches the titer of 1:96000 when the Aβ(1-11)E2₆₀ₘₑᵣ particles are used, and the titer 1:12000 when the Aβ(2-6)E2₆₀ₘₑᵣ particles are used. In the anti-beta amyloid antibodies obtained in these immunizations, a prevalence of the IgG 1 isotype with respect to the IgG2a isotype is observed, an isotype ratio that is indicative of a TH2 immune response. The proteins of the invention then enable to induce an antibody response against beta amyloid, with a high antibody titer, and a prevalence of the IgG1 isotype over the IgG2a isotype, indicative of a TH2 immune reaction. The proteins of the invention are useful for the development of vaccines against Alzheimer's disease and against amyloid deposition diseases.

To generate the virus-like particles Aβ(1-11)E260 mer and Aβ(2-6)E260 mer the authors exploited an antigen presentation system based on the pyruvate dehydrogenase complex of Geobacillus stearothermophilus (Domingo G. J., Perham R. N., EP1244775; WO0142439). Two of the enzymes that comprise it, E1 and E3, assemble on the surface of a large protein frame formed by the E2 enzyme, a dihydrolipoyl acyltransferase. The C-terminal catalytic domain of each E2 monomer is able to self-assemble in trimers, which in turn aggregate to form a 60 mer with icosahedral symmetry. The 60mer has molecular weight above 1.5 MDa and a diameter of 24 nm, viewable with the electronic microscope. Starting from denaturizing conditions, E2 can be renaturized in vitro to form the 60mer, with no need for chaperonins. An effective refolding of E2 can be obtained, with formation of 60mers, even when extraneous peptides or proteins are inserted at the N-terminal of the catalytic domain instead of the natural peripheral domains of E2. Then, the core domain of E2, (E2DISP: PGPAAAEEKAAPAAAKPATTEGEFPETREKMSGIRRAIAKAMVHSKHTAPHVTLMDE ADVTKLVAHRKKFKAIAAEKGIKLTFLPYVVKALVSALREYPVLNTSIDDETEEIIQKH YYNIGIAADTDRGLLVPVIKHADRKPIFALAQEINELAEKARDGKLTPGEMKGASCTIT NIGSAGGQWFTPVINHPEVAILGIGRIAEKPIVRDGEIVAAPMLALSLSFDHRMIDGAT AQKALNHIKRLLSDPELLLMEA (SEQ ID NO. 7), appropriately engineered, can expose up to 60 pairs of an exogenous polypeptide on the surface of a scaffold with high molecular weight (Domingo G J, et al., 2001).

The authors engineered E2DISP to obtain the protein Aβ(2-6)E2 (MAEFRHPGPAAAEEKAAPAAAKPATTEGEFPETREKMSGIRRAIAKAMVHSKHTAP HVTLMDEADVTKLVAHRKKFKAIAAEKGIKLTFLPYVVKALVSALREYPVLNTSIDD ETEEIIQKHYYNIGIAADTDRGLLVPVIKHADRKPIFALAQEINELAEKARDGKLTPGE MKGASCTITNIGSAGGQWFTPVINHPEVAILGIGRIAEKPIVRDGEIVAAPMLALSLSFD HRMIDGATAQKALNHIKRLLSDPELLLMEA, SEQ ID NO.8), which expresses at the N-terminal, immediately after the initial methionine, the epitope 2-6 of the beta amyloid 1-42 (DAEFRHDSGYEVHHQKLVFFAEDVGSNKGAIIGLMVGGVVIA, SEQ ID NO. 9) i.e. the amino acidic sequence AEFRH (SEQ ID NO. 5), and to obtain the protein Aβ(1-11)E2 (MDAEFRHDSGYEPGPAAAEEKAAPAAAKPATTEGEFPETREKMSGIRRAIAKAMVH SKHTAPHVTLMDEADVTKLVAHRKKFKAIAAEKGIKLTFLPYVVKALVSALREYPVL NTSIDDETEEIIQKHYYNIGIAADTDRGLLVPVIKHADRKPIFALAQEINELAEKARDGK LTPGEMKGASCTITNIGSAGGQWFTPVINHPEVAILGIGRIAEKPIVRDGEIVAAPMLAL SLSFDHRMIDGATAQKALNHIKRLLSDPELLLMEA, SEQ ID NO. 10), which expresses at the N-terminal, immediately after the initial methionine, the epitope 1-11 of the beta amyloid, i.e. the amino acidic sequence DAEFRHDSGYE (SEQ ID NO. 6).

The proteins Aβ(2-6)E2 and Aβ(1-11)E2, in purified form, spontaneously formed in vitro the virus-like particles Aβ(2-6)E2₆₀ₘₑᵣ e Aβ(1-11)E2₆₀ₘₑᵣ.

Therefore, the object of the invention is a multimeric protein molecule wherein each monomer comprises the sequence SEQ ID NO. 6 or an amino acidic sequence included in the sequence SEQ ID NO. 6 and an amino acidic sequence that constitutes the catalytic domain of the E2 monomer of the pyruvate dehydrogenase.

Preferably, the sequence comprised in the SEQ ID NO. 6 is the SEQ ID. NO. 5.

Preferably the monomer E2 of the pyruvate dehydrogenase is that of Geobacillus stearothermophilus.

More preferably the catalytic domain of the E2 monomer comprises the amino acidic sequence of SEQ ID NO. 7.

In one embodiment the multimeric protein molecule of the invention is a 60mer.

Preferably, the molecule is able to induce an antibody response against the beta amyloid peptide.

Yet more preferably, the multimeric protein molecule of the invention is for medical use.

In particular, for use in the treatment and/or prevention of neurodegenerative diseases.

Preferably for use in the treatment and/or prevention of Alzheimer's disease and/or of amyloid deposition diseases.

An object of the invention is the use of the multimeric protein molecule of the invention for the preparation of a medicament for the treatment and/or prevention of neurodegenerative diseases.

In particular, for the treatment and/or prevention of Alzheimer's disease and/or of amyloid deposition diseases.

An object of the invention is a pharmaceutical and/or immunizing composition comprising the molecule of the invention and pharmacologically acceptable excipients and/or diluents.

An additional object of the invention is a method for immunizing a patient comprising the administration of the molecule of the invention in which an antibody response to the beta amyloid protein is obtained.

Preferably, the antibody response has a prevalence of IgG 1. More preferably, the antibody response to the beta amyloid protein is of the TH2 type.

In the present invention, the beta amyloid peptide comprises the aggregate as well as the pre-aggregate and non aggregate form.

An embodiment of the invention is now described, premising that said description is not restrictive with respect to variations a person having ordinary skill in the related art may implement. We shall then present some examples that show the essential characteristics of the operation of the invention, specifying that said examples are not to be construed in a restrictive sense. The examples are illustrated by the following non limiting figures.

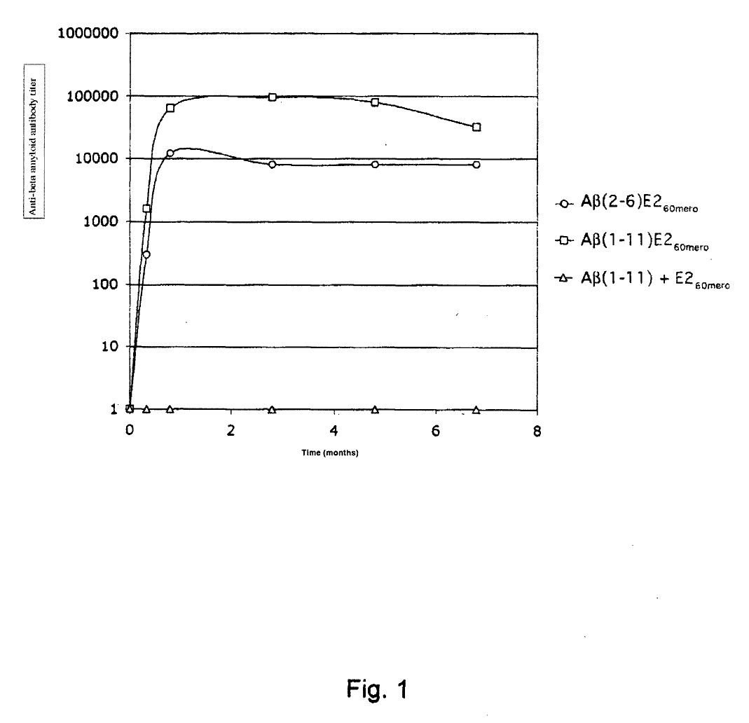

FIG. 1: The chart represents the variation over time of the average antibody titer observed in 3 groups of BALB/c mice, which on day 0 and on day 14 received an injection containing one of the following compositions: Aβ(2-6)E2₆₀ₘₑᵣ (group represented with the circle symbol), Aβ(1-11)E2₆₀ₘₑᵣ (group represented with the square symbol), the synthetic peptide Aβ(1-11) mixed with wild type E2₆₀ₘₑᵣ (group represented with the triangle symbol).

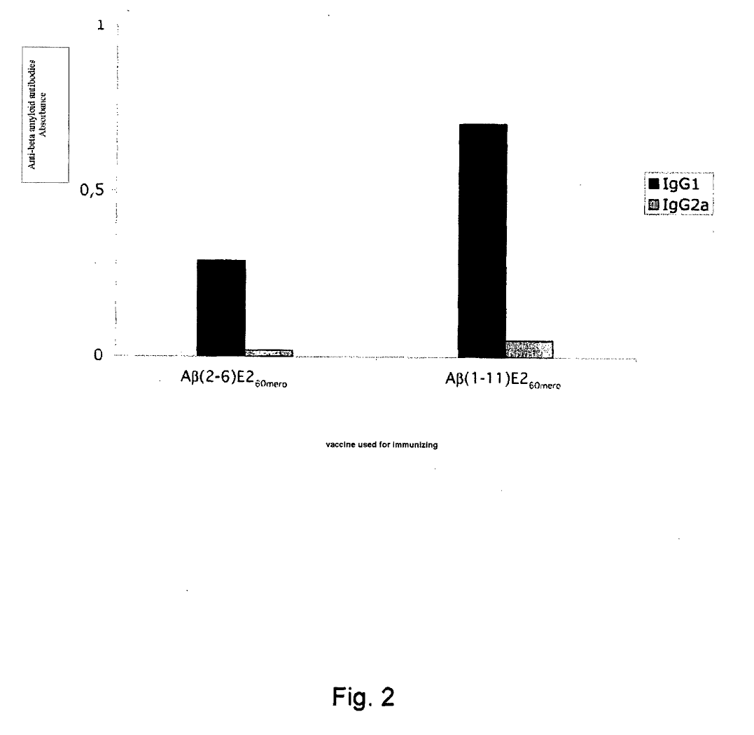

FIG. 2: The chart shows the absorbance obtained in an ELISA test that measured the anti IgG1 isotype or IgG2a antibodies that recognize the pre-aggregate beta amyloid peptide 1-42, in the serum of BALB/c mice subjected to an immunization and booster treatment with VLP Aβ(1-11)E2₆₀ₘₑᵣ or Aβ(2-6)E2₆₀ₘₑᵣ. In the illustrated experiment, the serums of the two groups of immunized mice, each comprising 5 animals, obtained 10 days after the second antigen injection, were mixed to form pools.

### METHODS

### Construction of the pET(2-6)E2 E pET(1-11)E2 Vectors

To obtain the expression of beta amyloid peptides as N-terminal fusions at the E2DISP core, the authors built, starting from the pETE2DISP vector, (Domingo G J, Orru' S, Perham R N, J Mol. Biol. 2001 Jan. 12; 305(2):259-67), the pET(2-6)E2 and pET(1-11)E2 VECTORS.

For this purpose, we used the following synthetic oligonucleotides:

SEQ ID NO. 1:
5'-CATGGCTGAATTCCGTCACC-3'
SEQ ID NO. 2:
5'-CCGGGGTGACGGAATTCAGC-3'
SEQ ID NO. 3:
5'-CATGGACGCTGAATTCCGTCACGACTCCGGTTACGAAC-3'
SEQ ID NO. 4:
5'-CCGGGTTCGTAACCGGAGTCGTGACGGAATTCAGCGTC-3'

SEQ ID NO. 1 and SEQ ID NO. 2 are complementary and encode the peptide Aβ(2-6); SEQ ID NO. 3 and SEQ ID NO. 4 are complementary and encode the peptide Aβ(1-11). Moreover, the oligonucleotides contain the sequences recognized by the NcoI and XmaI restriction enzymes. The complementary oligonucleotides were hybridized with each other (SEQ ID NO. 1 with SEQ ID NO. 2, SEQ ID NO. 3 with SEQ ID NO. 4) and inserted in the vector pETE2βISP digested with the enzymes NcoI and XmaI, by ligase with the enzyme T4 DNA ligase. The re-circularized plasmid was selected transforming E. coli TG1 cells, which were selected on LB-agar plates, containing ampicillin. The correctness of the sequences of the pET(2-6)E2 and pET(1-11)E2 plasmids was confirmed by DNA sequencing.

### Expression and Purification of the Aβ(2-6)E2 and Aβ(1-11)E2 Proteins

To produce the VLP Aβ(2-6)E2₆₀ₘₑᵣ and Aβ(1-11)E2₆₀ₘₑᵣ, E. coli BL21 (DE3) cells transformed with the pET(2-6)E2 or pET(1-11)E2 plasmids were grown in a LB medium containing 100 μg/ml of ampicillin at 37° C. up to an O.D. 0.6 (wavelength 600 nm), and then induced overnight at 30° C. with 1 mM IPTG (Isopropyl-beta-thiogalactopyranoside, Inalco, 1758-1400). The bacteria were harvested by centrifuging at 6000 rpm for 10 minutes, re-suspended in the A buffer (20 mM Tris-HCl pH8.5, 10 mM EDTA, 1 mM PMSF) containing 0.1 mg/ml lysozyme, placed in ice for 20 minutes, and lysated by sonication. The lysates were clarified by centrifuging at 10000 rpm for 1 hour, and the supernatant was subjected to fractioning with ammonium sulfate. The proteins precipitated at a saturation ranging between 35% and 65% were re-dissolved in the A buffer, dialyzed extensively against the same buffer, and applied on a Pharmacia Hiload 16/10Q-Sepharose HP anion exchange column, previously balanced with the B buffer (20 mM Tris-HCl pH 8.5, 1 mM EDTA). The Aβ(2-6)E2₆₀ₘₑᵣ and Aβ(1-11)E2₆₀ₘₑᵣ proteins were eluted with a linear gradient of 0-1 M in the B buffer, at a flow rate of 1 ml/minute on 200 ml. The fractions containing the recombinant proteins were joined together, concentrated using a Centriprep30 (Amicon), and loaded on a Pharmacia Superose6 gel filtration column balanced with 50 mM potassium phosphate pH 8.5. The proteins were eluted from the column at the characteristic elution volume of the 60mer. Protein concentration was determined with the Coomassie dye-binding method (Bradford assay). The purified VLP Aβ(2-6)E2₆₀ₘₑᵣ and Aβ(1-11)E2₆₀ₘₑᵣ were preserved at −80° C.

### Immunizations

The mice were immunized intraperitoneally with 200 μl of a 1:1 mixture of antigen and adjuvant, in particular with a such quantity of VLP as to contain 5 μg of the peptide of interest. In the first injection, the complete Freund's adjuvant (CFA) was used; in the subsequent injection, the incomplete Freund's adjuvant (IFA) was used. Blood was drawn from the tail 10 days after each immunization and at the indicated times.

### ELISA

The wells of a 96-well Nunc-Immuno plate were covered with streptavidin evaporation at 37° C. overnight. The wells were blocked with 0.5% BSA in 20 mM TrisHCl pH 7.3, 120 mM NaCl, covered with 50 ng of peptide biotinylate, incubated with mouse serum diluted in 0.25% BSA, 20 mM TrisHCl pH 7.3, 0.5M NaCl, 0.05% Tween 20, and detected with a mouse anti-IgG antibody conjugated with peroxidase (SIGMA A-2554). All incubations were done at 37° C. for 1 hour, and after each pass the wells were washed twice with EWB (20 mM TrisHCl pH 7.3, 130 mM NaCl, 0.05% Tween 20) and once with TBS (20 mM TrisHCl pH 7.3, 0.5M NaCl). The wells were incubated for 45 minutes at ambient temperature with 0.4 mg/ml O-phenylenediamine dihydrochloride dissolved in 30 mM citric acid, 70 mM Na₂HPO₄, 0.8 mM H₂O₂. Absorbance was read at 492 nm, after blocking color development with sulfuric acid 0.8M. Each serum was tested against the synthetic peptides 1-11 and 23-29 of the beta amyloid. When indicated, the serums were also tested against the pre-aggregated synthetic beta amyloid 1-42. The beta amyloid 1-42 was made to aggregate at 37° C. for 3 days, at the concentration of 1.1 mg/ml in NaCl 0.1M, NaHPO₄ 0.05M pH7.5. The wells of the ELISA plate were covered with 1 μg/well of sonicated amyloid aggregate, in PBS, by incubation at 4° C. for 4 hours.

To determine the isotype of the antibodies against the beta amyloid, the wells of an ELISA plate were covered with pre-aggregated beta amyloid, and the following BD Biosciences Pharmingen rat monoclonal antibodies were used: anti mouse IgM 550588, anti mouse IgG₁ 559626, anti mouse IgG₂ₐ 553391, anti mouse IgG_{2b} 550333, anti mouse IgG₃ 553401. To detect the biotinylated antibodies, streptavidin conjugated with peroxidase 554066 was used. The titer of a serum was defined as the highest dilution that gives a greater value of absorbance than double the background value obtained against an irrelevant antigen.

### EXAMPLES

### Example 1

After a single injection of the VLP Aβ(1-11)E2₆₀ₘₑᵣ of Aβ(2-6)E2₆₀ₘₑᵣ in CFA in two groups of Balb/c mice (5 mice per group) the authors of the present invention observed, after 10 days from the injection, a measurable anti-beta-amyloid response in all injected mice. The 5 control mice, injected with a mixture in CFA of the synthetic peptide Aβ(1-11) and of E2₆₀ₘₑᵣ wild type, not physically bound, did not produce antibodies against beta amyloid.

After a booster injection made 14 days after the first immunization using, for each mouse, the same antigen as the first injection, with the IFA adjuvant, the average anti-bet-amyloid titer measured after 10 days from the booster was 1:12000 in mice immunized with Aβ(2-6)E2₆₀ₘₑᵣ, and 1:64000 in mice immunized with Aβ(1-11)E2₆₀ₘₑᵣ, whilst not anti-beta-amyloid antibodies were produced in the control mice (FIG. 1).

### Example 2

The authors of the present invention analyzed the persistence of the anti beta amyloid antibodies in the serum of mice that received two injections of Aβ(1-11)E2₆₀ₘₑᵣ or Aβ(2-6)E2₆₀ₘₑᵣ. They observed that six months after the booster injection, anti-beta amyloid antibodies are still observed in all vaccinated mice, and the average anti-beta amyloid antibody titer is 1:8000 in mice vaccinated with Aβ(2-6)E2₆₀ₘₑᵣ and 1:32000 in those vaccinated with Aβ(1-11)E2₆₀ₘₑᵣ (FIG. 1).

### Example 3

The authors of the present invention measured the isotype of the anti-beta-amyloid antibodies obtained after an immunization and booster treatment with VLP Aβ(1-11)E2₆₀ₘₑᵣ or Aβ(2-6)E2₆₀ₘₑᵣ. Vaccination with the VLP Aβ(1-11)E2₆₀ₘₑᵣ or Aβ(2-6)E2₆₀ₘₑᵣ induces prevalently IgG1 antibodies, (indicating a TH2 response in mice), and a reduced quantity of IgG2a (indicating a TH1 response in mice) (FIG. 2).

### BIBLIOGRAPHY

Chackerian B., Rangel M., Hunter Z. and Peabody D. S. 2006. Vaccine. 24, 6321-6331.
Esposito M., et al., 2008. Mol. Immunol. 45, 1056-1062.
Gilman S., et al., 2005. Neurology. 64, 1553-1562.
Kim H. D., Jin J. J., Maxwell J. A. and Fukuchi K. 2007. Immunol Lett. 112, 30-38.
Morgan C., et al., 2004. Prog Neurobiol. 74, 323-349.
Morgan D. and Gitter B. D. 2004. Neurobiol Aging. 25, 605-608.
Weksler M. E., Gouras G., Relkin N. R. and Szabo P. 2005. Immunol Rev. 205, 244-256.
Domingo G. J., Perham R. N., EP1244775; WO0142439
Domingo G J, Orru' S, Perham R N, J Mol. Biol. 2001 Jan. 12; 305(2):259-67

1. A multimeric protein molecule wherein each monomer comprises the sequence SEQ ID NO. 6 or an amino acid sequence comprised in the sequence SEQ ID NO. 6, and an amino acidic sequence that constitutes the catalytic domain of the E2 monomer of the pyruvate dehydrogenase.

2. The multimeric protein molecule of claim 1 wherein the sequence comprised in the SEQ ID NO. 6 is the SEQ ID. NO. 5.

3. The multimeric protein molecule of claim 1 wherein the E2 monomer of pyruvate dehydrogenase is that of Geobacillus stearothermophilus.

4. The multimeric protein molecule of claim 3 wherein the catalytic domain of the E2 monomer comprises the amino acidic sequence of SEQ ID NO. 7.

5. The multimeric protein molecule of claim 1 being a 60mer.

6. The multimeric protein molecule of claim 1, able to induce an antibody response against the beta amyloid peptide.

7. (canceled)

8. A method for the treatment and/or prevention of neurodegenerative diseases, comprising administering the multimeric protein molecule of claim 1 to a patient in need thereof.

9. A method for the treatment and/or prevention of Alzheimer's disease and/or of amyloid deposition diseases, comprising administering the multimeric protein molecule of claim 1 to a patient in need thereof.

10. (canceled)

11. (canceled)

12. A pharmaceutical and/or immunizing composition comprising the molecule of claim 1 and pharmacologically acceptable excipients and/or diluents.

13. A method for immunizing a patient comprising the administration of the molecule of claim 1, wherein an antibody response to the beta amyloid protein is obtained.

14. The method of claim 13 wherein the antibody response has a prevalence of IgG1.

15. The method of claim 13 wherein the antibody response to the beta amyloid protein is of the TH2 type.


SEQUENCE LISTING (10 sequences)

SEQ ID NO: 1 - DNA, 20 bp, Artificial Sequence
  synthetic primer
catggctgaattccgtcacc

SEQ ID NO: 2 - DNA, 20 bp, Artificial Sequence
  synthetic primer
ccggggtgacggaattcagc

SEQ ID NO: 3 - DNA, 38 bp, Artificial Sequence
  synthetic primer
catggacgctgaattccgtcacgactccggttacgaac

SEQ ID NO: 4 - DNA, 38 bp, Artificial Sequence
  synthetic primer
ccgggttcgtaaccggagtcgtgacggaattcagcgtc

SEQ ID NO: 5 - protein, 5 aa, Artificial Sequence
  epitope beta amyloid 2-6
AEFRH

SEQ ID NO: 6 - protein, 11 aa, Artificial Sequence
  epitope beta-amyloid 1-11
DAEFRHDSGYE

SEQ ID NO: 7 - protein, 256 aa, Artificial Sequence
  core E2
PGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA

SEQ ID NO: 8 - protein, 262 aa, Artificial Sequence
  synthetic Ab(2-6)E2
MAEFRHPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA

SEQ ID NO: 9 - protein, 42 aa, Artificial Sequence [sequence omitted]

SEQ ID NO: 10 - protein, 268 aa, Artificial Sequence
  synthetic Abeta(1-11)E2
MDAEFRHDSGYEPGPAAAEEKAAPAAAKPATTEGEFPETREKMSGIRRAIAKAMVHSKHTAPHVTLMDEADVTKLVAHRKKFKAIAAEKGIKLTFLPYVVKALVSALREYPVLNTSIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLMEA